(19) United States
(12) Patent Application Publication
(10) Pub. No.: US 2026/0021158 A1
(43) Pub. Date: Jan. 22, 2026

(54) GRANULAR COMPOSITIONS COMPRISING A PEPTIDE AND USES THEREOF

(71) Applicant: TARSIER PHARMA LTD., Zichron Yaacov (IL)

(72) Inventors: Zohar MILMAN-LEVINSON, Rishpon (IL); Daphne HAIM-LANGFORD, Zichron Yaacov (IL); Natan HAYKIN, Eilat (IL); Hedyeh SAHEL, Abingdon, Oxfordshire (GB); Charlotte GOURMEL, Abingdon, Oxfordshire (GB); Hira Nisar AKHTAR, Abingdon, Oxfordshire (GB)

(21) Appl. No.: 19/100,988

(22) PCT Filed: Aug. 7, 2023
(86) PCT No.: PCT/IL2023/050818

§ 371 Date: Feb. 4, 2025

Related U.S. Application Data
(60) Provisional application No. 63/395,863, filed on Aug. 7, 2022.

(51) Int. Cl. A61K 38/07; A61K 9/00; A61K 47/34; A61K 47/54

(52) U.S. Cl. CPC A61K 38/07; A61K 9/0051; A61K 47/34; A61K 47/548

(57) ABSTRACT

Provided herein is a composition comprising solid particles or granules comprising Phosphorylcholine-tuftsin conjugate including a salt thereof, and ester end-capped poly(glycolide-co-lactide), wherein the Phosphorylcholine-tuftsin conjugate is an amorphous solid; is characterized by any one of: (i) average particle size less than 30 um, as determined by SEM; (ii) a powder XRD devoid of a corresponding peak having a net intensity peak height of above about 30 counts. Additionally, method for treating an ocular disease or disorder in a subject via intraocular administration of a therapeutically effective amount of the composition of the invention to the subject.

### CROSS-REFERENCE TO RELATED APPLICATIONS

This application claims the benefit of priority of U.S. Provisional Patent Application No. 63/395,863, titled “GRANULAR COMPOSITIONS COMPRISING A PEPTIDE AND USES THEREOF” filed Aug. 7, 2022, the contents of which are incorporated herein by reference in their entirety.

### FIELD OF INVENTION

This invention is directed to solid polymeric granules comprising a peptide and uses thereof, such as for prevention or treatment of an ocular disease or disorder in a subject and/or conditions associated therewith.

### BACKGROUND OF THE INVENTION

Ocular inflammation, an inflammation of any part of the eye, is one of the most common ocular diseases. Ocular inflammation refers to a wide range of inflammatory disease of the eye, one of them is uveitis. These diseases are prevalent in all age groups and may be associated with systemic diseases such as Crohn's disease, Behcet disease, Juvenile idiopathic arthritis and others. The inflammation can also be associated with other common eye symptoms such as dry eye and dry macular degeneration. Several drugs have the known side effect of causing uveitis and/or dry eye. The most common treatment for ocular inflammation, is steroids and specifically corticosteroids. However, these treatments have several known and sometimes severe side effects.

Dazdotuftide or Phosphorylcholine-tuftsin conjugate (PTC) is a bi-specific synthetic peptide molecule with immunomodulatory activities. It is composed of Tuftsin (Thr-Lys-Pro-Arg) a natural immunomodulating peptide produced by enzymatic cleavage of the Fc-domain of the heavy chain of IgG in the spleen. Phosphorylcholine (PC) is a small zwitterionic molecule secreted by helminths which permits helminths to survive in the host inducing a situation of immune tolerance as well as on the surface of some bacteria and apoptotic cells.

Solid formulations of Phosphorylcholine-tuftsin conjugate for direct administration to the eye are greatly needed. Specially, such solid formulations should be characterized by: a sufficient storage shelf-life, and by a gradual release of the active agent at the application site within a predetermined time period.

### SUMMARY OF THE INVENTION

In one aspect of the invention, there is provided a composition comprising a Phosphorylcholine-tuftsin conjugate including a salt thereof, wherein the Phosphorylcholine-tuftsin conjugate is represented by Formula 1:

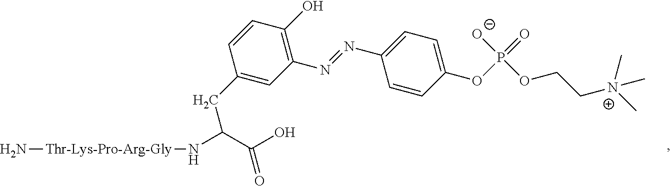

the conjugate is in a form of a particulate matter characterized an average particle size less than 300 um, as determined by SEM; and wherein the composition is characterized by a powder XRD being devoid of a corresponding peak of the Phosphorylcholine-tuftsin conjugate having a net intensity peak height above 400 counts.

In one embodiment, the salt thereof is a pharmaceutically acceptable salt, wherein the composition has a water content below 20%.

In one embodiment, the composition is a pharmaceutical composition comprising a pharmaceutically effective amount of the Phosphorylcholine-tuftsin conjugate and further comprising a pharmaceutically acceptable carrier.

In another aspect, there is provided a composition comprising a plurality of particles, wherein each of the plurality of particles is a solid particle comprising a mixture of poly(glycolide-co-lactide) and a Phosphorylcholine-tuftsin conjugate wherein: the plurality of particles is characterized by at least one dimension greater than 100 um; and the Phosphorylcholine-tuftsin conjugate is an amorphous solid as determined by XRD.

In one embodiment, a weight concentration of the Phosphorylcholine-tuftsin conjugate within the plurality of particles is between about 1 and about 50%; and wherein the Phosphorylcholine-tuftsin conjugate is in a form of a particulate matter characterized an average particle size less than 300 um, as determined by SEM.

In one embodiment, at least one of: (i) between 50% and 100% w/w of polymeric chains of the poly(glycolide-co-lactide) are ester end-capped; (ii) a weight ratio between polylactide and polyglycolide within the poly(glycolide-co-lactide) is at least 1:1; (iii) wherein the poly(glycolide-co-lactide) has an acid value of below 1 mg(KOH)/g; or any combination of (i)-(iii).

In one embodiment, the composition is characterized by a powder XRD being devoid of a corresponding peak of the Phosphorylcholine-tuftsin conjugate having a net intensity peak height above 400 counts.

In one embodiment, Phosphorylcholine-tuftsin conjugate is represented by Formula 1:

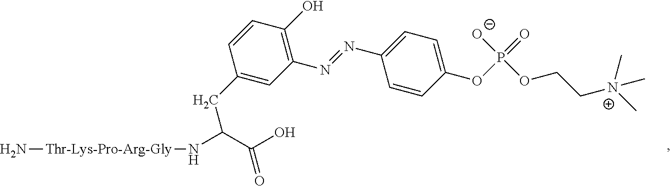

In one embodiment, the composition is characterized by density between 0.3 and 0.4 mg/mm3.

In one embodiment, the plurality of particles are ocular drug implant (ODIs).

In one embodiment, the ODI is in a form of an elongated particle characterized by at least one of: a length dimension between about 1 and about 10 mm; a width dimension between about 0.1 and about 0.8 mm; optionally wherein the ODI comprises a therapeutically effective amount of the Phosphorylcholine-tuftsin conjugate.

In another aspect, there is provided an ocular drug implant (ODI), wherein the ODI is a solid material comprising a mixture of poly(glycolide-co-lactide) and a peptide in a form of particulate matter; wherein: the ODI is characterized by at least one dimension greater than 100 um; and the particulate matter is characterized by an average particle size of at most about 100 um, as determined by SEM.

In one embodiment, a weight concentration of the peptide within the ODI is between about 1 and about 50%.

In one embodiment, at least one of: (i) a weight ratio between polylactide and polyglycolide within the poly(glycolide-co-lactide) is at least 50:50; (ii) at least 80% w/w of polymeric chains of the poly(glycolide-co-lactide) are ester end-capped; (iii) the poly(glycolide-co-lactide) has an acid value of below 1 mg(KOH)/g; or a combination of (i)-(iii).

In one embodiment, the peptide is a hydrophilic peptide characterized by an aqueous solubility of at least 10g/L; optionally, wherein the peptide is a phosphorylcholine-peptide conjugate.

In one embodiment, the ODI is substantially in a form of elongated particles, optionally characterized by at least one of: a length dimension between about 1 and about 10 mm; a width dimension between about 0.1 and about 0.8 mm.

In one embodiment, the ODI is an extrudate.

In one embodiment, the ODI is characterized by a substantial release of the Phosphorylcholine-tuftsin conjugate, or of the peptide in an aqueous medium.

In one embodiment, a weight ratio between polylactide and polyglycolide within the poly(glycolide-co-lactide) is at least 50:50; wherein at least 80% w/w of polymeric chains of the poly(glycolide-co-lactide) are ester end-capped; and wherein the substantial release comprises a cumulative release of at least 30% of the initial amount of the peptide or of the Phosphorylcholine-tuftsin conjugate within a time period ranging between about 2 and about 30 days.

In one embodiment, a particle size of at least 80% by weight of the particulate matter has a particle size between about 5 and about 100 um, as determined by SEM.

In one embodiment, an average particle size of the particulate matter is between about 10 and about 50 um, as determined by SEM.

In one embodiment, the ODI comprises a therapeutically effective amount of the peptide.

In another aspect, there is provided a method for treating an ocular disease or disorder in a subject, the method comprises intraocularly administering a therapeutically effective amount of the composition of the inveniton or of the ODI of the inveniton to the subject.

In one embodiment, the therapeutically effective amount comprises a daily dose of the phosphorylcholine-tuftsin conjugate or of the peptide between 0.01 and 100 μg.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE FIGURES

The invention will now be described in relation to certain examples and embodiments with reference to the following illustrative figures so that it may be more fully understood. In the drawings:

FIG. 1 is a powder XRD of pristine unmilled PTC pellets (upper graph, black), and of the grinded PTC powder according to the invention (lower graph, red). “PTC” refers to a compound of Formula 1, as disclosed herein.

FIGS. 2A-2B are SEM images of pristine unmilled PTC particulate matter (2A) and of PTC particulate matter according to the invention (2B).

FIG. 3A are SEM images of an exemplary rod-shaped solid particle of the invention (i.e. ocular drug implant, ODI) obtained by extrusion of Resomer RG 752 S and grinded PTC powder according to the invention (9% w/w drug loading). Left image presents a cross-section, right image presents a surface of the rod-shaped solid particle.

FIG. 3B is a graph showing an aqueous cumulative % release of PTC from an ODI composed of ester end-capped PLGA (Resomer RG 752 S).

FIG. 3C is an image of an exemplary rod-shaped solid particle of the invention (ODI).

FIG. 3D is a graph showing aqueous cumulative release of PTC from exemplary rod-shaped solid particles of the invention (ODI) with 9% drug loading composed of ester end-capped PLGA (Resomer RG 752 S), as compared to a similar particle composed of non-end capped Resomer RG 752 H.

### DETAILED DESCRIPTION OF THE INVENTION

The present invention, in some embodiments thereof, relates to a solid composition (c.g., an ophthalmic composition) comprising inter alia phosphorylcholine-tuftsin conjugate, and to a method for treating medical conditions by administering the composition to a subject in need thereof. The present invention, in further embodiments thereof, relates to an ophthalmic composition for ocular administration and to use thereof in the treatment of medical conditions in a subject in need thereof.

The present invention, in some embodiments thereof is based on a surprising finding that poly(glycolide-co-lactide) (PLGA)-based solid particles (i.c. ODIs) comprising phosphorylcholine-tuftsin conjugate (PTC), as disclosed herein, exhibited improved release profile, as compared to microspheres composed of the similar constituents. Specifically, the inventors surprisingly found that ester-end-capped PLGA (termed herein as Resomer 752S), when utilized in the solid particles of the invention exhibited superior drug loading efficiency and aqueous release profile (showing a sustained release, as demonstrated herein), as compared to a similar acid-terminated polymer (i.c., non-end capped PLGA, termed herein as Resomer 752H). Additionally, the inventors surprisingly found that by utilizing a PTC with reduced average particle size of below about 30 um, resulted in homogenous solid particles and in improved sustained release profile therefrom. In contrast, similar solid particles comprising unmilled (pristine) solid PTC with an average particle size between about 30 and about 100 um resulted in a burst release profile.

The term “phosphorylcholine tuftsin conjugate” as used herein, refers to a phosphorylcholine moiety covalently linked to tuftsin or to a tuftsin derivative, optionally via a spacer. The term “phosphorylcholine tuftsin conjugate” as used herein further encompasses any salt (e.g. a pharmaceutically acceptable salt) and any isotope thereof).

As used herein, the term “tuftsin” refers to a tetra-peptide (threonine-lysine-proline-arginine, TKPR; SEQ ID NO: 1). In some embodiments, the PTC is or comprises tuftsin (i.e. a peptide having an amino acid sequence as defined in SEQ ID NO: 1) covalently bound to a phosphorylcholine moiety via a side chain (e.g. phosphate group is bound directly to a side-chain of threonine, or to a side chain of lysine). In some embodiments, the PTC is or comprises tuftsin (i.e. a peptide having an amino acid sequence as defined in SEQ ID NO: 1) covalently bound to a phosphorylcholine moiety via the amino terminus (e.g. phosphate group is bound directly to the amino group, generating phosphoramidate). In some embodiments, the PTC is or comprises tuftsin (i.e. a peptide having an amino acid sequence as defined in SEQ ID NO: 1) covalently bound to a phosphorylcholine moiety via the amino terminus (e.g. phosphate group is bound directly to the amino group, generating phosphoramidate). In some embodiments, the PTC is or comprises tuftsin (i.e. a peptide having an amino acid sequence as defined in SEQ ID NO: 1) covalently bound to a phosphorylcholine moiety via the carboxy terminus (e.g. carboxy group is bound directly to the phosphate, generating a phosphorylated carboxylate).

The term “phosphorylcholine moiety” encompasses phosphoryl choline, i.e.

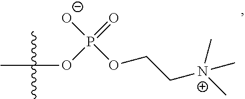 [SMILES: *OP(=O)([O-])OCC[N+](C)(C)C]

and a derivative of phosphorylcholine. The term “derivative of phosphorylcholine” as used herein, refers to any compound that is based on phosphorylcholine. n some embodiments, the derivative retains the immunomodulatory effects of phosphorylcholine. In some embodiments, the phosphorylcholine derivative is a derivative comprising phosphorylcholine.

In some embodiments, the derivative of phosphorylcholine is selected from: phenyl-phosphorylcholine, a substituted phenyl-phosphorylcholine (e.g. aminophenyl-phosphorylcholine, nitrophenyl-phosphorylcholine, halophenyl-phosphorylcholine, hydroxyphenyl-phosphorylcholine, alkylphenyl-phosphorylcholine) and 12-(3-iodophenyl) dodecyl-phosphocholine among others. Each possibility is a separate embodiment of the invention.

In some embodiments, the derivative of phosphorylcholine is represented by Formula 2:

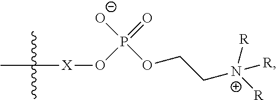

wherein each R independently is an optionally substituted alkyl (e.g. methyl, or any C1-10, or C2-C10 alkyl); and wherein X is a spacer. In some embodiments, X comprises a small molecule such as a natural and/or unnatural amino acid(s), a C5-C10cycloalkylene, optionally substituted C1-C6alkylene, —C(═O)-C1-C6alkylene, optionally substituted C6-C10arylene, aryl (or heteroaryl)-azo, heteroaromatic ring(s), a carbocyclyl; a bond (such as an amide bond, an ester bond, azo bond, a thioester bond, a disulfide bond, —N—C(═O)-, —C(═O)N—, CONR′-, —CNNR′-, —CSNR′-, —NC(═O)O—, —NC(═S)O—, —NC(═S)N—, —SO2-, —SO—, -SR′, —C(═O)-, —OC(═O)-, —OC(═O)O—, —OC(═S)O—, an d—OC(═S)N—; —S—C(═O)), a glycol of formula -(RO)x-, wherein R represents C1-C10 alkyl; and x is an integer ranging between 1 and 10, or any combination thereof.

In some embodiments, X is or comprises a linear or a branched chain. In some embodiments, X comprises a backbone comprising a linear or a branched chain. In some embodiments, X comprises a cyclic (aromatic or aliphatic) backbone.

In some embodiments, the derivative of phosphorylcholine is represented by Formula 2, wherein R is methyl.

In some embodiments, X is

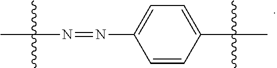 [SMILES: *N=Nc1ccc(*)cc1]

In some embodiments, the spacer has a MW less than 500 Da, less than 400 Da, less than 300 Da, less than 200 Da, less than 100 Da, or between 30 and 100 Da, between 30 and 200 Da, between 30 and 300 Da, including any range between.

In some embodiments, the spacer is between 1 and 50, between 1 and 100, between 2 and 100, between 2 and 80, between 2 and 60, between 5 and 50, between 10 and 50, between 10 and 40, between 2 and 30, between 2 and 20, between 2 and 10, between 1 and 5, between 5 and 10, between 5 and 15, between 5 and 25, between 5 and 50 single C-C bonds long, including any range in between.

The term “tuftsin derivative” refers to tuftsin (TKPR, SEQ ID NO: 1) attached to at least two additional amino acids which are independently selected. Non-natural amino acids, preferably non-charged and non-polar non-natural amino acids such as β-alanine-6-aminohexanoic acid and 5-aminopentanoic acid, may also be comprised in the tuftsin derivative. In some embodiments, the tuftsin derivative is TKPR (X1) (X2), wherein X1 is an amino acid selected from Gly, Ala, Val, Thr, Leu, Ile, and Met; and wherein X2 is an amino acid selected from Tyr, Trp, Phe, Cys, Ser, Thr, Gly, Ala, Val, Thr, Leu, Ile, and Met.

In some embodiments, the tuftsin derivative is a peptide comprising TKPR and retaining the immunomodulatory effects of tuftsin. A derivative is not merely a fragment of the polypeptide, nor does it have amino acids replaced or removed (an analog), rather it may have additional amino acid residues and/or modification made to the polypeptide, such as a post-translational modification.

In some embodiments, the tuftsin derivative is Threonine-Lysine-Proline-Arginine-Glycine-Tyrosine (TKPRGY, SEQ ID NO: 2).

In some embodiments, the term “moiety” as used herein refers to a part of a molecule, which lacks one or more atom(s) compared to the corresponding molecule. The term “moicty”, as used herein, may further relate to a part of a molecule that may include either whole functional groups or parts of functional groups as substructures (e.g. an amino group lacking a hydrogen, a phosphate group lacking hydrogen or hydroxy, amino acid residue, etc). The term “moiety” further means part of a molecule that exhibits a particular set of chemical and/or pharmacologic characteristics which are similar to the corresponding molecule.

The terms “linked” or “attached” as used herein refer to a bond between at least two molecules or moieties such that they are a single molecule. In some embodiments, the bond is a chemical bond. In some embodiments, the bond is a covalent bond. According to the principles of the present invention, the natural and non-natural amino-acids comprised in the tuftsin derivative are adjacent and attached to one another, while the at least one phosphorylcholine derivative is attached to the at least one tuftsin derivative either directly or indirectly via a spacer. In some embodiments, the at least one phosphorylcholine or derivative thereof is linked to the N-terminus of at least one tuftsin or derivative thereof. In some embodiments, the at least one phosphorylcholine or derivative thereof is linked to the C-terminus of at least one tuftsin or derivative thereof.

In some embodiments, the phosphorylcholine-tuftsin conjugate comprises one or more phosphorylcholine moiety(s) attached to a tuftsin derivative. In certain embodiments, the phosphorylcholine moiety (i.c., a derivative of phosphorylcholine represented by Formula 2) is covalently bound to a tuftsin derivative. In some embodiments, covalently bound is via a side chain of the tuftsin derivative. In some embodiments, covalently bound is via a side chain of Tyr (c.g. via an azo bond bound to the side chain of Tyr). In some embodiments, the phosphorylcholine-tuftsin conjugate is represented by Formula 1, below.

In one aspect of the invention, there is provided a composition comprising a Phosphorylcholine-tuftsin conjugate (PTC) including any salt thereof, wherein the PTC is in a form of a particulate matter characterized by any one of: (i) average particle size less than 300 um, or less than 200 um, as determined by SEM; and (ii) a powder XRD devoid of a corresponding peak of the PTC having a net intensity peak height of above about 400 counts. In some embodiments, the PTC is as disclosed herein.

In another aspect of the invention, there is provided a composition comprising a peptide including any salt thereof, wherein the peptide is in a form of a particulate matter characterized by any one of: (i) average particle size less than 300 um, or less than 200 um, as determined by SEM; and (ii) a powder XRD devoid of a corresponding peak of the peptide having a net intensity peak height of above about 400 counts. In some embodiments, the peptide is a peptide-phosphorylcholine conjugate, as disclosed herein for PTC. In some embodiments, the peptide is a peptide-phosphorylcholine conjugate comprising the phosphorylcholine moiety bound to (i) a side chain of the peptide (e.g. Lys, or Tyr side chain), and/or (ii) to the C-terminus or N-terminus of the peptide.

In some embodiments, the peptide is between 3 and 50, between 3 and 10, between 3 and 20, between 3 and 5, between 5 and 10 amino acid residues long, including any range between.

As used herein, the terms “peptide”, “polyaminoacid”, “polypeptide” and “protein” are used interchangeably and refer to a polymer of amino acid residues. In some embodiments, the peptide of the invention is or comprises a therapeutic peptide sequence. The term “therapeutic peptide sequence” refers to any peptide sequence configured for inducing a therapeutic effect within a subject (e.g., treating, preventing, reducing symptoms of a disease, etc.). Further, the term “therapeutic peptide sequence” encompasses any polyamino acid sequence capable of modifying the activity, functionality, survival, fitness, appearance, structure, development, behavior, or any combination thereof, of a cell. In some embodiments, the therapeutic sequence is capable of binding an intracellular target (e.g. enzyme), so as to control (upregulate, or downregulate) the activity of the intracellular target.

The terms “peptide”, “polyaminoacid”, “polypeptide” and “protein” as used herein encompass native peptides, peptide derivatives such as beta peptides, peptidomimetics (typically including non-peptide bonds or other synthetic modifications,) and the peptide analogs peptoids and semi-peptoids or any combination thereof. In another embodiment, the terms “peptide”, “polyaminoacid” and “protein” apply to amino acid polymers in which at least one amino acid residue is an artificial chemical analog of a corresponding naturally occurring amino acid.

The term “derivative” or “chemical derivative” includes any chemical derivative of the polypeptide having one or more residues chemically derivatized (or chemically modified) by reaction on the side chain or on any functional group within the peptide. Such derivatized molecules include, for example, peptides bearing one or more protecting groups (e.g., side chain protecting group(s) and/or N-terminus protecting groups), and/or peptides in which free amino groups have been derivatized to form amine hydrochlorides, p-toluene sulfonyl groups, carbobenzoxy groups, t-butyloxycarbonyl groups, acetyl groups or formyl groups. Free carboxyl groups may be derivatized to form amides thereof, salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and Dab, Daa, and/or ornithine(O)may be substituted for lysine.

In addition, a peptide derivative can differ from the natural sequence of the peptide of the invention by chemical modifications including, but are not limited to, terminal-NH2 acylation, acetylation, or thioglycolic acid amidation, and by amidation of the terminal and/or side-chain carboxy group, e.g., with ammonia, methylamine, and the like. Peptides can be either linear, cyclic, or branched and the like, having any conformation, which can be achieved using methods known in the art.

The term “amino acid” as used herein means an organic compound containing both a basic amino group and an acidic carboxyl group. Included within this term are naturally occurring amino acids, protected amino acids (e.g., comprising one or more protecting groups at the carboxyl, at the amine, and/or at the side chain of the amino acid), unusual, non-naturally occurring amino acids (such as D-amino acids), as well as amino acids which are known to occur biologically in free or combined form but usually do not occur in proteins. Included within this term are modified and unusual amino acids, such as those disclosed in, for example, Roberts and Vellaccio (1983) The Peptides. 5:342-429. Modified, unusual or non-naturally occurring amino acids include, but are not limited to, D-amino acids, hydroxylysine, 4-hydroxyproline, N-Cbz-protected aminovaleric acid (Nva), ornithine (O), aminooctanoic acid (Aoc), 2,4-diaminobutyric acid (Abu), homoarginine, norleucine (Nle), N-methylaminobutyric acid (MeB), 2-naphthylalaninc (2Np), aminoheptanoic acid (Ahp), phenylglycine, β-phenylproline, tert-leucine, 4-aminocyclohexylalanine (Cha), N-methyl-norleucine, 3,4-dehydroproline, N,N-dimethylaminoglycine, N-methylaminoglycine, 4-aminopipetdine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, cyano-propionic acid, 2-benzyl-5-aminopentanoic acid, Norvaline (Nva), 4-O-methyl-threonine (TMe), 5-O-methyl-homoserine (hSM), tert-butyl-alanine (tBu), cyclopentyl-alanine (Cpa), 2-amino-isobutyric acid (Aib), N-methyl-glycine (MeG), N-methyl-alanine (MeA), N-methyl-phenylalanine (MeF), 2-thienyl-alanine (2Th), 3-thienyl-alanine (3Th), O-methyl-tyrosine (YMe), 3-Benzothienyl-alanine (Bzt) and D-alanine (DAl).

The term “polyaminoacid” further encompasses random polymers (i.e., devoid of a specific amino acid sequence within the entire composition and include a random population of polymers of different lengths and of different sequences) and polypeptides having a specific amino acid sequence). The terms “peptide sequence” and “amino acid sequence” are used herein interchangeably. In some embodiments, the peptide sequence is or comprises D-amino acid sequence. In some embodiments, at least 70%, at least 80%, at least 90%, at least 95% of the amino acids within the peptide sequence are in D-configuration. In some embodiments, the amino acids within the peptide sequence are in D-configuration.

In some embodiments, the composition of the invention (e.g., ODI) comprises the peptide in a form of a particulate matter, as disclosed herein.

In another aspect of the invention, there is provided a composition comprising a PTC including a salt thereof, wherein the PTC is represented by Formula 1:

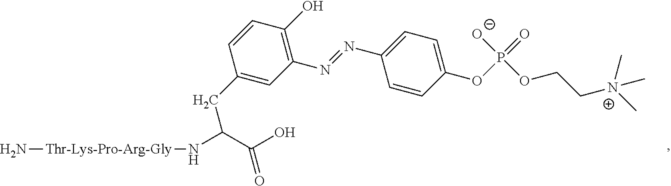

the PTC is an amorphous solid; and the PTC is in a form of a particulate matter characterized by any one of: (i) average particle size less than 300 um, or less than 200 um, as determined by SEM; (ii) a powder XRD devoid of a corresponding peak having a net intensity peak height of above about 400 counts. In some embodiments, the PTC is an amorphous powder.

In some embodiments, the average particle size of the particulate matter is between 10nm and 300 um, between 10 nm and 200 um, between 10 nm and 180 um, between 10 nm and 150 um, between 10 nm and 100 um, between 20 nm and 100 um, between 10 nm and 80 um, between 10 nm and 75 um, between 10 nm and 1 um, between 10 nm and 10 um, between 100 nm and 10 um, between 100 nm and 1 um, between 100 nm and 5 um, between 10 nm and 500 um, between 100 nm and 500 um, between 300 nm and 10 um, between 300 nm and 5 um, between 300 nm and 1 um, between 300 nm and 500 um, between 300 nm and 800 um, between 500 nm and 10 um, between 500 nm and 1 um, between 10 nm and 100 nm, between 100 nm and 500 nm, between 500 nm and 800 um, between 800 nm and 30 um, between 800 nm and 5 um, between 5 and 30 um, between 1 and 30 um, between 1 and 10 um, between 1 and 75 um, between 1 and 150 um, between 1 and 100 um, between 1 and 50 um, between 1 and 60 um, between 10 and 30 um, including any range between, wherein the average particle size is as determined by SEM. In some embodiments, the particle size of at least 80% by weight of the particulate matter has a particle size between about 5 and about 100 um, between about 20 and about 100 um, between about 20 and about 80 um, between about 20 and about 60 um, between about 20 and about 50 um, between about 20 and about 40 um, as determined by SEM.

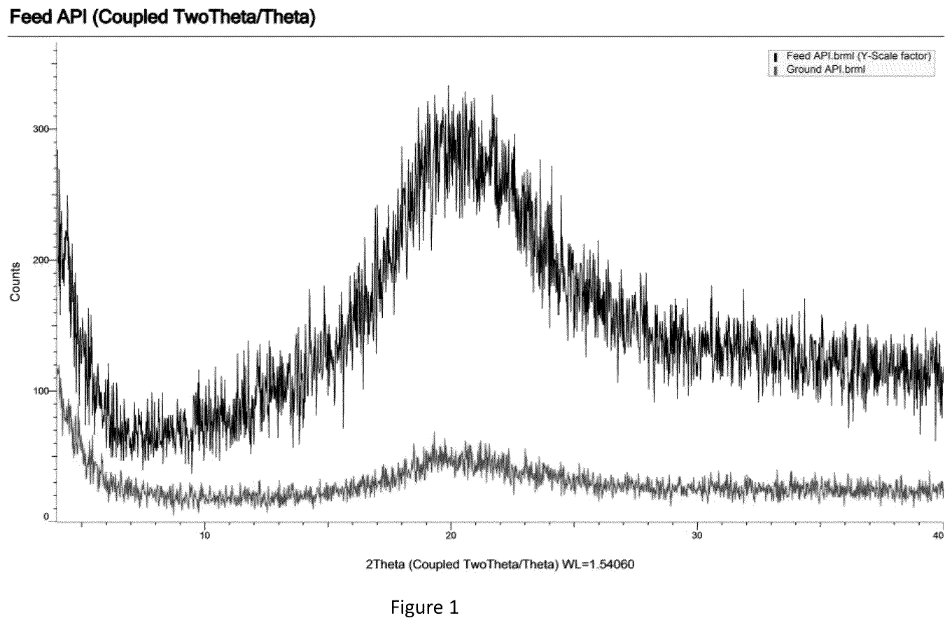

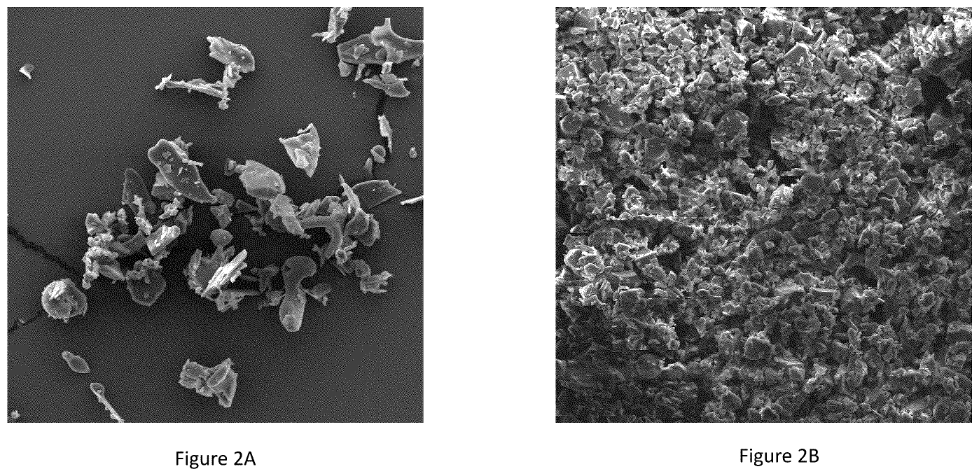

In some embodiments, the particulate matter is an amorphous powder. In some embodiments, the PTC (particulate matter) is characterized by a powder XRD (X-Ray diffractogram) being substantially devoid of a corresponding peak. In some embodiments, the XRD of the PTC is characterized by a corresponding peak being significantly lower than the corresponding peak of the pristine powderous PTC. The term “pristine” refers to the PTC obtained by lyophilization of the purified (or crude) PTC solution. The inventors observed that the pristine PTC is substantially characterized by an average particle size of about 200 um, and is further in a form of irregularly shaped pellets. Furthermore, the pristine PTC is characterized by a small corresponding peak at powder XRD (see FIG. 1, upper diffractogram), whereas the PTC of the invention is substantially devoid of any significant corresponding XRD peak, as visualized by FIG. 1 (lower diffractogram in red).

In some embodiments, the particle size of the particulate matter is as described above, wherein the particulate matter is amorphous, as characterized by powder XRD of the PTC being devoid of a corresponding peak (i.e. peak of the PTC) having a net intensity of above 400 counts, above 300counts, above 200 counts, above 100 counts, about 50 counts, about 40 counts, about 30 counts, about 20 counts, about 10 counts, about 5 counts, including any range between. In some embodiments, the particle size of the particulate matter is as described above, wherein the powder XRD of the PTC is substantially devoid of a corresponding peak having a gross intensity of above about 200 counts, above about 150 counts, above about 100 counts, above about 80 counts, above about 60 counts, above about 50 counts, above about 40 counts, above about 30 counts, above about 20 counts, above about 10 counts, including any range between. In some embodiments, the corresponding peak of the PTC as measured by powder XRD, has a peak height (net-intensity) between 1 and 200 counts, between 1 and 100 counts, between 1 and 300 counts, between 1 and 50 counts, between 1 and 40 counts, between 1 and 30 counts, between 1 and 20 counts, between 1 and 10 counts, including any range between, wherein the peak height refers to normalized peak intensity.

The inventors surprisingly found that the PTC (e.g. PTC of Formula 1) in a form of the particulate matter described herein is highly superior over pristine PTC, especially in terms of incorporation thereof into solid particles of the invention (ODI), so as to result in ODI with an optimal loading of the active agent (PTC) and further characterized by a sustained release profile of the active agent.

In some embodiments, the salt of the PTC is a pharmaceutically acceptable salt. Pharmaceutically acceptable salts are well-known in the art, including inter alia within alkali metal salts, alkaline earth metal salts and/or ammonium salts, as well as halide (e.g., chloride), citrate, acetate, trifluoroacetate, phosphate, borate, lactate and the like salts and mixtures thereof. In some embodiments, the peptide (e.g. PTC) disclosed herein is a pharmaceutical grade active agent (e.g., characterized by chemical purity above 97%). In some embodiments, the entire constitutes of the composition and/or ODI of the invention are substantially chemically pure compounds.

In some embodiments, the composition of the invention is a pharmaceutical composition comprising a pharmaceutically effective amount of the PTC and further comprising a pharmaceutically acceptable carrier. In some embodiments, the composition of the invention is a pharmaceutical composition comprising a pharmaceutically effective amount of a peptide and further comprising a pharmaceutically acceptable carrier.

In some embodiments, the composition of the invention is formulated for ocular administration. In some embodiments, the pharmaceutical composition is an ophthalmic composition. In some embodiments, the terms “ophthalmic composition” and “pharmaceutical composition” are used herein interchangeably. In some embodiments, the pharmaceutical composition is formulated for ocular administration. In some embodiments, the composition of the invention comprises the phosphorylcholine-tuftsin conjugate as the only pharmaceutically active ingredient. In some embodiments, the composition of the invention is substantially devoid of any additional pharmaceutically active ingredient. In some embodiments, the composition of the invention is substantially devoid of any additional peptide. In some embodiments, the composition of the invention is substantially devoid of any additional anti-inflammatory agent.

In another aspect, there is provided a composition comprising a plurality of particles, wherein each of the plurality of particles is a solid particle comprising a poly(glycolide-co-lactide) (PLGA) and the Phosphorylcholine-tuftsin conjugate of the invention. In some embodiments, the solid particle is an ODI. The terms “solid particle(s)” and “ODI” are used herein interchangeably.

In some embodiments, PLGA and PTC are mixed together within the solid particle. In some embodiments, PLGA and PTC are in a form of a mixture within the solid particle. In some embodiments, PLGA and PTC are in a form of a homogenous mixture within the solid particle. In some embodiments, the solid particle is a composite material comprising or consisting essentially of PLGA and PTC. In some embodiments, the solid particle is or consist essentially of a mixture of PLGA and PTC. In some embodiments, “consists essentially of” encompasses between 80 and 100%, between 80 and 99%, between 90 and 99%, between 90 and 100%, between 92 and 99%, between 93 and 99%, between 95 and 99%, between 95 and 97% between 93 and 100%, between 95 and 100%, between 97 and 99%, between 97 and 100% by dry weight of the solid particle.

In some embodiments, the PLGA is in a form of a matrix. In some embodiments, the mixture comprises of PTC particles embedded or incorporated within a PLGA matrix. In some embodiments, the mixture comprises of PTC particles enclosed by the PLGA matrix.

As used herein, the term “matrix” refers to one or more layers of polymeric chains that are randomly (and/or have an ordered distribution) distributed therewithin. Further to the PTC particles, matrix may further include any materials incorporated within and/or interposed between the layers. In some embodiments, the matrix comprises randomly oriented polymeric chains. In some embodiments, each polymeric chain within the matrix is in contact with at least one additional polymeric chain. In some embodiments, the polymeric chains are randomly distributed within the matrix, to obtain a three-dimensional mesh structure comprising a void space between the chains. In some embodiments, the polymeric chains are randomly distributed within the matrix thus forming an intertwined polymeric mesh optionally a plurality of pores (or void space). In some embodiments, the matrix is an intertwined matrix composed of randomly distributed polymeric chains, and is characterized by low porosity, as disclosed herein. In some embodiments, the matrix is substantially devoid of polymeric chains aligned or oriented in a specific direction.

In some embodiments, (i) the PLGA is substantially ester end-capped; and/or (ii) a weight ratio between polylactide and polyglycolide within the poly(glycolide-co-lactide) is between 50:50 and 95:5, including any range between. In some embodiments, the ester end-capped PLGA refers to an alkylated terminal carboxy group of the polymer (e.g., terminal glycolate alkylation). In some embodiments, the terminal carboxy group is alkylated by a C1-C10 alkyl, so as to obtain a terminal C1-C10 ester (e.g., methyl ester). In some embodiments, at least 70%, at least 80%, at least 85%, at least 90%, at least 95%, at least 97%, at least 99% w/w, or between 60 and 95%, between 70 and 95%, between 80 and 95%, between about 80 and about 90%, between about 85 and about 90% by weight of the entire polymeric chains of PLGA are ester end-capped.

In some embodiments, at least 50%, at least 70%, at least 80%, at least 85%, at least 90%, at least 95%, at least 97%, at least 99% w/w, or between 60 and 95%, between 70 and 95%, between 50 and 95%, %, between 50 and 99%, between 80 and 95%, between about 80 and about 90%, between about 85 and about 90% by weight of the entire polymeric chains of PLGA are ester end-capped.

In some embodiments, the ester end-capped PLGA has at most 20%, at most 15%, at most 10% of an acid value, as compared to an acid value of the same polymer which is not ester end-capped (i.e. free acid polymer). In some embodiments, the acid value of the ester end-caped PLGA is below 1 mg KOH/gr of PLGA.

In some embodiments, the PLGA comprises less than 20%, less than 10%, less than 5%, less than 3%, less than 1% by weight of non-end-capped PLGA (e.g., PLGA having a terminal carboxy group) by total weight of PLGA.

In some embodiments, the PLGA is characterized by an average molecular weight (Mw) between 5000 and 10000, between 10000 and 20000, between 10000 and 15000, between 5000 and 15000, between 7000 and 20000 Da, including any range between.

In some embodiments, the solid particle disclosed herein is in a solid state at a temperature below the melting point of PLGA (e.g., below 200° C., or below 150° C.).

In another aspect, the ODI of the invention comprises a biodegradable polymer and a peptide, wherein the peptide is in a form of a particulate matter, as disclosed herein and is mixed with the biodegradable polymer within the entire volume of the ODI, and wherein a weight concentration of the peptide within the ODI is as disclosed herein (between 1-50% w/w). In some embodiments, the biodegradable polymer is a polyester. In some embodiments, the polyester is non-end capped. In some embodiments, the polyester is ester end-capped. In some embodiments, the polyester comprises any one of a polycaprolactone, poly-ε-caprolactone (PCL), a polyglycolide, a polylactide, poly-1-lactide (PLLA), poly-d,1-lactide (PLA),a polyglycolide, polylactic polycaprolactone (PCL), polyhydroxyalkanoate, polyhydroxybutyrate, polyethylene adipate, polybutylene succinate, poly(3-hydroxybutyrate-co-3-hydroxyvalerate), polyethylene terephthalate (PET), polybutylene terephthalate, polyethylene naphthalate (PEN), including any copolymer or any combination thereof. In some embodiments, the biodegradable polymer is PLGA, and the peptide is a PTC, as disclosed herein.

In some embodiments, the composition of the invention comprising the solid particles disclosed herein, is a pharmaceutical composition. In some embodiments, the composition of the invention is composed essentially of pharmaceutical grade constituents. In some embodiments, the composition of the invention is for use in the treatment of an ocular disease or disorder, or a condition associated therewith, via intra-ocular administration of a pharmaceutically effective amount of the composition to the subject. In some embodiments, the pharmaceutically effective amount of the composition comprises a pharmaceutically effective amount of PTC.

In some embodiments, the solid particle (ODI) is a melt granulated unit pharmaceutical dosage form of the PTC of the invention. In some embodiments, the solid particle (ODI) comprises a therapeutic amount of the peptide (i.e. a therapeutic peptide such as PTC, as disclosed herein) per each ODI ranging between 0.01 and 200 μg, between 0.01 and 100 μg, between 1 and 100 μg, between 0.1 and 100 μg, between 10 and 100 μg, between 100 and 200 ug, between 50 and 200 μg, between 20 and 100 μg, between 20 and 200 μg, including any range or value therebetween.

ODIs refer to intraocular implants, which are well-known in the art. ODIs are made to be implanted in order to regulate the drug efflux and, as a result, lengthen the time that the disease condition is under control. ODIs may release high medicament concentrations in the intended locations via site-specific implantation. Additionally, ODIs increases patient compliance, minimizes parenteral treatment pain, and sustains the drug concentration in the therapeutic window by a continuous controlled release of the loaded medication. ODIs can be administered to a subject via implantation such as by intravitreal injection, intracameral injection, and subconjunctival injection.

In some embodiments, the solid particles are extruded (or hot melt extruded) particles. In some embodiments, the solid particle is characterized by at least one dimension greater than 50 um, greater than 100 um, greater than 200 um, greater than 300 um, greater than 400 um, greater than 500 um, greater than 0.5 mm, greater than 1 mm, including any range between. The term “dimension” refers to any one of: a length dimension, a width dimension (e.g., cross-section or diameter/radius), or both. In some embodiments, the term “dimension” refers to an average particle size in the composition of the invention. In some embodiments, the solid particles is substantially devoid of microspheres, such as particles (e.g., spherical particles) with an average particle size between 1 and 50 um, between 1 and 30 um, between 1 and 20 um, including any range between. In some embodiments, the particles are in a form of granules. In some embodiments, the particles are in a form of solid granules. In some embodiments, the particles or granules are substantially uniformly shaped. In some embodiments, the particles or granules are characterized by a substantially uniform size distribution, loading of the PTC, or both.

In some embodiments, a weight concentration of the conjugate within the solid particles (or within the composition) is between about 1 and about 50%, between about 1 and about 40%, between about 1 and about 30%, between about 5 and about 50%, between about 5 and about 30%, between about 1 and about 20%, between about 5 and about 20%, between about 1 and about 10%, between about 10 and about 50%, between about 10 and about 40%, between about 20 and about 50%, including any range between. The inventors successfully manufactured exemplary ODI of the invention with up to about 20% w/w loading of PTC (between about 2.5 and about 20% loading). Currently, the inventors presume that it is possible to obtain a significantly higher loading of PTC in the ODI up to at least 40% by weight.

In some embodiments, the composition of the invention is composed essentially of ester end-capped PLGA and the PTC of the invention. In some embodiments, the solid particles of the invention is composed essentially of ester end-capped PLGA and the PTC of the invention.

In some embodiments, the PTC is in an amorphous state within the solid particle, as determined by XRD. In some embodiments, the PTC within the solid particles substantially retains the particle size of the powderous PTC. In some embodiments, the PTC within the solid particle(s) substantially retains the amorphous state of the powderous PTC. In some embodiments, the composition of the invention (or plurality of particles disclosed herein) is characterized by substantially the same XRD, as the powderous PTC disclosed herein.

In some embodiments, the PTC is substantially homogenously distributed within the solid particles of the invention. In some embodiments, the PTC is substantially homogenously distributed within the composition of the invention. Homogenous distribution can be determined by HPLC, such as by measuring PTC concentration in 3 or more different probes sampled from the composition of the invention. The composition is considered as homogenous, if the standard deviation of the PCT concentration values of the samples is below 10%.

In some embodiments, a weight ratio between polylactide and polyglycolide within the poly(glycolide-co-lactide) is at least 50:50, at least 60:40, at least 70:30, at least 80:20, at least 90:10, between 50:50 and 95:5, between 50:50 and 90:10, between 50:50 and 85:15, between 50:50 and 80:20, between 60:40 and 95:5, between 60:40 and 90:10, between 60:40 and 85:15, between 60:40 and 80:20, between about 70:30 and about 80:20, including any range between. Without being limited to any particular theory, the inventors postulated (based on experimental data) that PLGA with a weight ratio between polylactide and polyglycolide above 1:1 (i.e. a weight excess of polylactide over polyglycolide) and more particularly above 60:40, such as about 75:25, or about 65:35 results in solid particles of the invention (ODI) being characterized by a preferable sustained release profile of the active agent (PTC) therefrom. PLGA with a weight ratio between polylactide and polyglycolide below 1:1 (e.g. 10:90, 25:75, or between 5:95 and 50:50, including any range between) can be utilized for the manufacturing of ODIs with fast drug release.

In some embodiments, the solid particles (ODI) have an elongated shape. In some embodiments, each of the solid particles of the invention is characterized by an elongated shape. In some embodiments, the solid particles are substantially characterized by a rod shape, a bar shape, a needle shape, cylindrical shape, elliptical shape, etc. A skilled artisan will appreciate that the shape of each of the solid particles may slightly or substantially vary from a specific geometrical shape. Accordingly, the solid particles may have a rod-like shape, a bar-like shape, a needle-like shape, a cylinder-like shape, or ellipse-like shape, meaning that the actual shape of the particle has some deviations (e.g., at least 10%, at least 50%, or more deviation) from a perfect geometrical shape. In some embodiments, the solid particles is substantially devoid of hollow shaped particles.

The solid particles may be of any shape, such as cubic shape, a rectangular shape, a prism shape, a conical shape, etc.

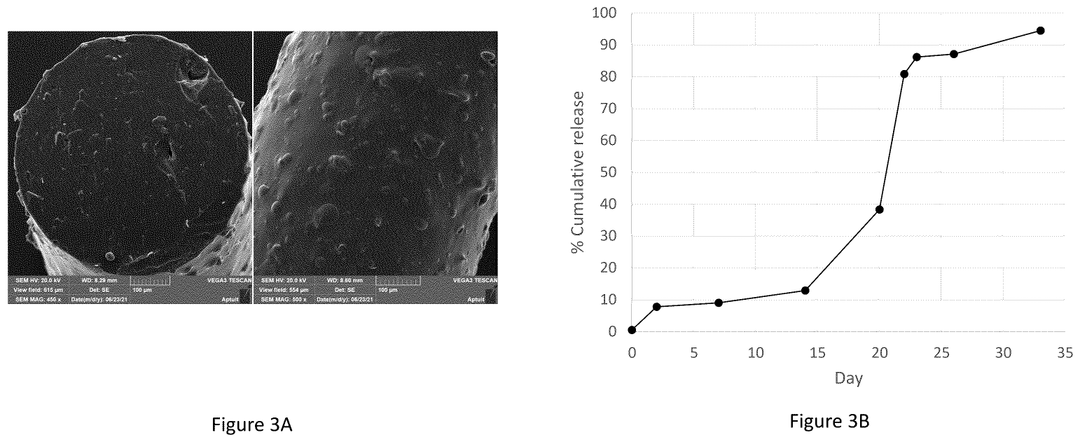

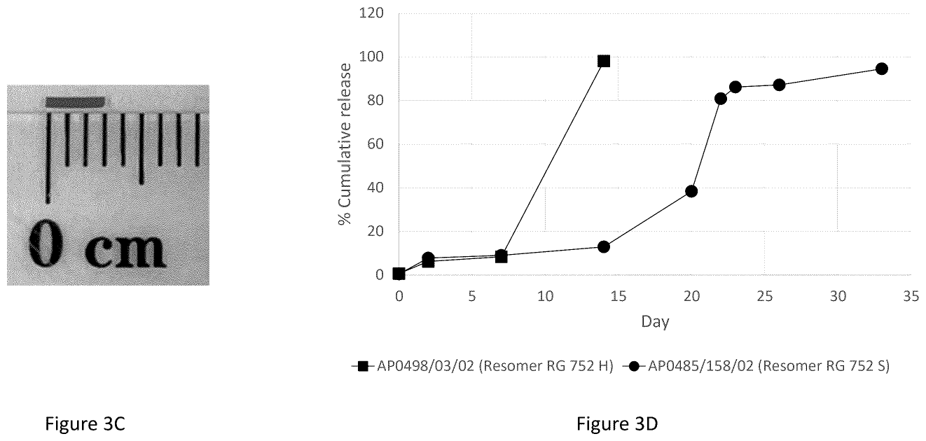

In some embodiments, the solid particles are uniformly shaped particles, wherein at least 70%, at least 80%, at least 90%, at least 95%, at least 97%, at least 99% of the solid particles have substantially (e.g., with a size deviations of up to 20%, or up to 10%, including any range between) the same shape, including any range between. In some embodiments, the solid particles (ODIs) are substantially in a form of rod-shaped particles or cylinder-shaped particles (see FIG. 3A).

In some embodiments, the solid particles is characterized by a length dimension of at least 100 um, at least 500 um, between about 0.5 and about 100 mm, between about 0.5 and about 50 mm, between about 0.5 and about 30 mm, between about 0.5 and about 10 mm, between about 1 and about 100 mm, between about 1 and about 50 mm, between about 1 and about 30 mm, between about 1 and about 10 mm, between about 1 and about 5 mm, including any range between. In some embodiments, the length dimension is greater than 100 mm.

In some embodiments, the solid particles is characterized by a width dimension between about 0.05 and about 2 mm, about 0.05 and about 1.5 mm, about 0.05 and about 1 mm, about 0.1 and about 2 mm, about 0.1 and about 1 mm, about 0.1 and about 0.6 mm, about 0.2 and about 2 mm, about 0.2 and about 1 mm, about 0.2 and about 0.8 mm, about 0.1 and about 0.8 mm, including any range between. In some embodiments, the width dimension of the solid particles is predetermined by the inner cross-section of a mean (e.g., catheter) for intra-ocular delivery of the particles to the subject. A skilled artisan will appreciate, that for the intra-ocular delivery the particles of the invention have to be compatible with the means for intra-ocular delivery, thus without being limited, the particle's width dimension has to be less than 0.8, preferably less than 0.6 mm.

The terms “length dimension” and “width dimension” as used herein, each independently refer to the average value (e.g., number average), as determined by SEM or as measured by a caliper. Methods for determining average length or average width of the solid particles in a given sample are well-known in the art. In an exemplary embodiment, average length or average width of the elongated particles can be determined by SEM or other microscopes, using an appropriate SEM or other microscopes image processing software, or by measuring each particle individually using a caliper. Exemplary elongated solid particle of the invention is presented in FIG. 3C.

In some embodiments, the solid particles is characterized by an aspect ratio is 1, between about 2 and about 100, between about 2 and about 10, between about 2 and about 20, between about 2 and about 30, between about 2 and about 50, between about 5 and about 100, between about 5 and about 50, between about 5 and about 20, between about 5 and about 30, including any range between. In some embodiments, the solid particles of the invention is characterized by a length dimension, a width dimension, and optionally by an aspect ratio, as described herein.

In some embodiments, the solid particles is substantially non-porous, characterized by a porosity of less than 20%, less than 10%, less than 5%, including any range between.

In some embodiments, the solid particles and/or the composition comprising the solid particles is characterized by a density between 0.2 and 0.6 mg/mm³, 0.3 and 0.45 mg/mm³, between about 0.3 and about 0.4 mg/mm³, between 0.30 and 0.33 mg/mm³, between 0.32 and 0.35 mg/mm³, between 0.35 and 0.40 mg/mm³, between 0.40 and 0.45 mg/mm³, between 0.31 and 0.33 mg/mm³, including any rang between,

In another aspect of the present invention, there is provided a composition comprising a plurality of particles, wherein each of the plurality of particles is a solid particle comprising a mixture of PLGA and a peptide in a form of a particulate matter; wherein the plurality of particles within the composition are characterized by at least one dimension greater than 50 um, greater than 100 um, greater than 200 um, greater than 300 um, greater than 400 um, greater than 500 um, greater than 0.5 mm, greater than 1 mm, between 0.1 and 10 mm, between 0.1 and 3 mm, between 0.1 and 2 mm, including any range between; and wherein an average particle size of the particulate matter is at most about 300 um, at most about 200 um, at most about 150 um, is at most about 70 um, or at most about 30 um, or between 10 and 200 um, between 10 and 150 um, between 10 and 100 um, between 10 and 70 um, between 10 and 50 um, including any rang between; wherein the average particle size of the particulate matter is determined by SEM. In some embodiments, the solid particles are elongated particles, characterized by a width dimension greater than 50 um, greater than 100 um, greater than 200 um, greater than 300 um, greater than 400 um, greater than 500 um, greater than 0.5 mm, greater than 1 mm between 0.1 and 3 mm, between 0.1 and 2 mm, between 0.5 and 1 mm, between 0.5 and 2 mm, including any range between. In some embodiments, the solid particles are as described hereinabove.

In some embodiments, any one of: the particle size of the peptide, a weight concentration of the peptide within the particles/composition, the chemical composition of PLGA and other physico-chemical parameters of the particles is/are as described herein for the solid particles comprising PTC.

In some embodiments, the peptide is a hydrophilic peptide. In some embodiments, the hydrophilic peptide is characterized by an aqueous solubility (i.e. in an aqueous solution devoid of an organic solvent) of at least 10 g/L, at least 20 g/L, at least 50 g/L, at least 70 g/L, at least 100 g/L, at least 200 g/L, at least 300 g/L, at least 500 g/L, including any range between at a temperature between 20 and 30° C. In some embodiments, the peptide is or comprises PTC, as disclosed herein.

In some embodiments, the solid particles disclosed herein are hot-molten particles. In some embodiments, the solid particles disclosed herein are extruded particles, i.e., obtained via a hot-melt extrusion.

In some embodiments, the corresponding peak of the solid particles (ODI) of the invention, is characterized by substantially higher XRD peak height (net-intensity) of the peptide as compared to a control, wherein the control is a non-extruded mixture containing the same constituents. In some embodiments, the corresponding peak (i.e. the XRD peak of the peptide) of the ODI is lower than the control by at least 10 times, at least 8 times, at least 5 times, at least 2 time, at least 1.5 times, including any range in between.

In some embodiments, the corresponding peak of the composition of the invention, is characterized by substantially higher XRD peak height (net-intensity) as compared to a control, wherein the control is a non-extruded composition containing the constituents as the composite, and wherein the XRD peak refers to the corresponding peak of the peptide. In some embodiments, the composition is characterized by a corresponding XRD peak of the peptide being lower by up to 500%, up to 90%, up to 80%, up to 70%, up to 60%, up to 50%, up to 40%, up to 30%, up to 20%, up to 20%, up to 15%, up to 10% and up to 5% including any range between, when compared to the corresponding peak of the control.

In some embodiments, the solid particles (ODI) disclosed herein are characterized by a substantial release of the peptide (e.g., a hydrophilic peptide such as PTC) therefrom. In some embodiments, the term “release” refers to the release of the peptide into an aqueous medium (e.g., at a temperature between 10 and 50° C., and/or pH of the aqueous medium between about 5 and about 8, including any range between). In some embodiments, the term “substantial release” refers to the release of at least at least 10%, 30%, at least 50%, at least 70%, at least 90% of the initial peptide loading within the solid particles or within the composition of the invention including any range between. In some embodiments, the solid particles disclosed herein are characterized by a delayed release onset, so that the substantial release occurs after 1 day, after 2days, after 3, 4, or 5 days or after a time period ranging between 2 and 20, between 2 and 10, between 5 and 20, between 5 and 15, between 5 and 10 days upon contacting the solid particles with the aqueous medium. It is postulated that the release profile obtained in the aqueous medium is indicative with respect to in-vivo release at the application site within the subject (e.g., intraocular release).

In some embodiments, the term “substantial release” refers to the cumulative release of at least 10%, at least 30%, at least 50%, at least 70%, at least 90% of the initial loading of the peptide within a time period ranging between about 1 and about 90 days, between about 1 and about 5 days, between about 1 and about 10 days, between about 2 and about 5 days, between about 2 and about 10 days, including any range between. In some embodiments, the cumulative release is measured from the release onset, wherein the release onset is as described herein. In some embodiments, the release profile of the peptide is substantially gradual or sustained, and is devoid of a burst release, wherein the release refers to the cumulative release, as described herein. Exemplary release profile is demonstrated in FIG. 3B. In some embodiments, release of the peptide can be determined by HPLC.

In another aspect, there is a method for treating an ocular disease or disorder in a subject, the method comprises intraocularly administering a therapeutically effective amount of the composition of the invention (e.g., ophthalmic composition comprising the solid particles disclosed herein) to the subject.

In some embodiments, the eye of the subject is afflicted with an inflammation. In some embodiments, the inflammation is an ocular inflammation. In some embodiments, the eye of the subject is afflicted with any one of dry eye, dry macular degeneration, diabetic macular edema, and post operation inflammation. In some embodiments, ocular inflammation is uveitis.

In some embodiments, the method is for treating or preventing a disease or a disorder associated with ocular inflammation.

As used herein, the term “ocular inflammation” refers to any inflammation of any part of the eye. In some embodiments, the inflammation is of the middle layer of the eye. In some embodiments, the inflammation is uveitis. In some embodiments, the ocular inflammation comprises dry eye or dry macular degeneration. In some embodiments, the ocular inflammation is associated with another disease.

Non-limiting examples of systemic diseases which can result in ocular inflammation are Crohn's disease, Bechet disease, and Juvenile idiopathic arthritis. In some embodiments, the ocular inflammation is associated with an adverse reaction to a drug or environmental trigger. Non-limiting examples of such drugs include Rifabutin, quinolones, vaccines and allergens. In some embodiments, the ocular inflammation is associated with post operation inflammation. Non-limiting examples of such include post-cataract surgery, laser eye surgery and corneal transplantation.

As used herein, the terms “treatment” or “treating” of ocular inflammation encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life. In some embodiments, treating ocular inflammation comprises at least one of preventing the onset of ocular inflammation, attenuating the progress of ocular inflammation and inhibiting the progression of ocular inflammation.

In some embodiments, treating comprises reducing inflammation. In some embodiments, treating comprises reducing abnormal inflammation. In some embodiments, treating comprises reducing inflammation in an eye of the subject.

In some embodiments, treating comprises reducing secretion of at least one pro-inflammatory cytokine.

In some embodiments, reducing comprises at least a 10%, 20%, 30%, 40%, 50%, 60%, 70%, 80%, 90%, 95%, 99% or 100% reduction. Each possibility represents a separate embodiment of the invention. It will be understood by one skilled in the art that each cytokine need not be reduced by the same amount. Some cytokines may be reduced by more than others.

In another aspect of the invention, there is a method for enhancing ocular bioavailability of a phosphorylcholine-tuftsin conjugate in a subject, comprising administering to an eye of the subject the ophthalmic composition of the invention.

In some embodiments, enhancing ocular bioavailability is by at least 10% compared to a control, wherein the control is as disclosed hereinbelow. In some embodiments, enhancing is by at least 10%, at least 20%, at least 30%, at least 40%, at least 50%, at least 60%, at least 70%, at least 80%, at least 90%, at least 95%, at least 99%, at least 100%, at least 200%, at least 500%, at least 1000%, at least 5000%, at least 10.000%, at least 100.000%, including any range or value therebetween.

In some embodiments, the method is for prolonging residence time of phosphorylcholine-tuftsin conjugate on or within an eye (e.g., cornea and/or aqueous humor and/or vitreous humor and/or choroid). In some embodiments, prolonging is for a time period ranging between 1 and 90 days, between 1 and 5 d, between 2 and 10 d, between 5 and 20 d, between 2 and 20 d, between 2 and 30 d, between 5 and 50 d, including any value there between, wherein the term “prolonging” is as compared to a control composition which is composed of the same constituents as the composition of the inveniton, wherein the PTC is in a form of substantially spherical microparticles (e.g., with an average particle size between about 10 and about 100 um). In some embodiments, the control composition comprises the same constituents as the composition of the inveniton, wherein the polymer is a non-end capped PLGA (see FIG. 3D).

In some embodiments, enhancing ocular bioavailability comprises increasing concentration of the phosphorylcholine-tuftsin conjugate in an aqueous humor and/or vitreous humor of the eye. In some embodiments, increasing is by at least 10%, at least 20%, at least 30%, at least 40%, at least 50%, at least 60%, at least 70%, at least 80%, at least 90%, at least 95%, at least 99%, at least 100%, at least 200%, at least 500%, at least 1000%, at least 5000%, at least 10.000%, at least 100.000%, including any range or value therebetween, as compared to the control composition.

In some embodiments, the subject is selected from a human subject and an animal subject.

As used herein, the terms “administering”, “administration”, and like terms refer to any method which, in sound medical practice, delivers a composition containing the peptide (i.e., the active agent) to a subject in such a manner as to provide a therapeutic effect. In some embodiments, the administering is ocular or intraocular. In some embodiments, the administering is via a catheter, such as an ocular catheter, or any other mean for ocular or intraocular delivery of solid particles disclosed herein. In some embodiments, the administering is via intravitreal administration. In some embodiments, the administering step is repeated for example 2, 3, 4, 5, or 10 times within 24 h up to 1 year.

In some embodiments, the amount (dose) of a composition to be administered will, of course, be dependent on the subject being treated, in the medical condition being treated for, the severity of the affliction, the manner of administration, the judgment of the prescribing physician, etc.

In some embodiments, the daily dose (i.e. the amount of the phosphorylcholine-tuftsin conjugate per day) is between 0.01 and 200 μg, between 0.01 and 100 μg, 50 and 2000 μg, between 200 and 2000 μg, between 50 and 100 μg, between 100 and 200 μg, between 200 and 300 μg, between 300 and 400 μg, between 400 and 500 μg, between 500 and 600 μg, between 600 and 700 μg, between 700 and 800 μg, between 800 and 900 μg, between 900 and 1000 μg, between 1000 and 1100 μg, between 1100 and 1300 μg, between 1300 and 1500 μg, between 1500 and 1800 μg, between 1800 and 2000 μg, including any range or value therebetween.

In some embodiments, the daily dose (i.e. the amount of the phosphorylcholine-tuftsin conjugate per day) is between 0.01 and 100 μg, or between 1 and 100 μg.

According to an embodiment of the present invention, the pharmaceutical composition described herein above is packaged in a packaging material and identified in print, in or on the packaging material, for use in the treatment of a disease or disorder, as described herein.

According to another embodiment of the present invention, the pharmaceutical composition is packaged in a packaging material and identified in print, in or on the packaging material, for use in monitoring a disease or disorder, as described herein.

Products of the present invention may, if desired, be presented in a pack or dispenser device, such as an U.S. Food and Drug Administration (FDA) approved kit, which may contain one or more unit dosage forms containing the disclosed composition. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the FDA for prescription drugs or of an approved product insert.

In some embodiments, the kit contains a single dosage form, wherein the dosage form contains a daily dose of the disclosed composition. In some embodiments, the kit contains a plurality of dosage forms. In some embodiments, the kit contains a plurality of dosage forms, wherein the plurality of dosage forms are equivalent to a daily dose of the disclosed composition.

In some embodiments, the method is for extending the release period on or within the eye of the phosphorylcholine-tuftsin conjugate. In some embodiments, phosphorylcholine-tuftsin conjugate is slowly released. In some embodiments, phosphorylcholine-tuftsin conjugate is released in a controlled manner. In some embodiments, the method is for inducing a delayed release onset of PTC, wherein delayed is as described herein. In some embodiments, the method is for inducing a sustained release of the phosphorylcholine-tuftsin conjugate.

In this context, the term “controlled manner” indicates that the drug is released substantially constantly. Herein, the term “constantly” may refer to a time duration as described herein above (between 1 and 90 days).

It is understood that the composition of the present invention may be administered in conjunction with other drugs, including other anti-inflammatory drugs.

In another aspect of the invention, there is provided a method for manufacturing the solid particle of the invention, the method comprises extruding a powderous composition or a kit, comprising the peptide (e.g. the PTC of the invention) characterized by an average particle size as disclosed herein; and the PLGA as disclosed herein, wherein the PLGA is characterized by an average particle size between 10 nm and 100 um, between 10 nm and I um, between 10 nm and 10 um, between 100 nm and 10 um, between 100 nm and 1 um, between 100 nm and 5 um, between 10 nm and 100 um, between 100 nm and 80 um, between 100 nm and 70 um, between 100 nm and 60 um, between 100 nm and 50 um, between 100 nm and 30 um, between 100 nm and 20 um, between 100 nm and 10 um, between 100 nm and 5 um, between 100 nm and 1 um, between 300 nm and 10 um, between 300 nm and 5 um, between 300 nm and 1 um, between 300 nm and 500 um, between 300 nm and 800 um, between 500 nm and 10 um, between 500 nm and 1 um, between 500 nm and 50 um, between 500 nm and 100 um, between 10 nm and 100 nm, between 100 nm and 500 nm, between 500 nm and 80 um, between 800 nm and 30 um, between 800 nm and 5 um, between 5 and 30 um, between 1 and 30 um, between 1 and 10 um, between 10 and 30 um, between 10 and 60 um, between 10 and 80 um, between 30 and 60 um, between 30 and 50 um, including any range between; wherein extruding is performed under suitable conditions. In some embodiments, each of the peptide (e.g. the PTC of the invention) and the PLGA is introduced separately into the extruder. In some embodiments, the peptide (e.g. the PTC of the invention) and the PLGA are mixed together, so as to obtain a composition, wherein the composition is subsequently introduced into the extruder. In some embodiments, extrusion is performed at a temperature between 60 and 80° C., between 60 and 70° C., between 65 and 75° C., between 70 and 80° C., including any range between.

In some embodiments, the method is for manufacturing the particles or granules of the invention (i.e. ODI). In some embodiments, the method is for obtaining an extrudate. In some embodiments, the extrudate is further undergoes shaping via a thermal molding process selected from extrusion, injection, hot blown film, molding (e.g., cast molding, compression molding, rotational molding) or any combination thereof. In some embodiments, the extrudate is shapeable or processable so as to obtain the solid particles of the invention characterized by a predetermined shape and/or dimension(s).

In some embodiments, there is provided a method for shaping the particles of the invention, the method comprises extruding the extrudate in an extruder, by utilizing an extrusion die characterized by a predetermined shape and/or dimension.

In some embodiments, the method comprises a preliminary step of milling or grinding any one of the pristine peptide (e.g. the pristine PTC) and the pristine PLGA, so as to obtain the powderous composition or a kit, comprising the peptide and the PLGA characterized by a particle size as disclosed herein. In some embodiments, the pristine PLGA and the pristine peptide are milled or grinded simultaneously or separately. In some embodiments, milling or grinding is accompanied by cooling the milling or grinding chamber.

In some embodiments, the components composing the composition of the invention (and/or the extrudate) can be processed via an extrusion process. In some embodiments, physical properties (such as particle size, chemical composition, ratio between the peptide and the PLGA, thermal stability of the peptide) of the components composing the composition of the invention and/or of the extrudate are compatible with, or suitable for an extrusion process.

In some embodiments, the solid particles (or granules) of the invention are substantially stable under suitable storage conditions (e.g., a temperature as described herein, and exposure to ambient atmosphere). In some embodiments, the solid particles (or granules) of the invention are substantially stable at a temperature of between 30 and 60° C., between −50 and 60° C., between 0 and 10° C., between 10 and 30° C., between 30 and 50° C., including any range therebetween.

As used herein the term “stable” refers to the ability of the solid particles (or granules) of the invention to substantially maintain its structural, physical and/or chemical properties. In some embodiments, the solid particles (or granules) of the invention are referred to as stable, when it substantially maintains its structure (e.g., shape, and/or a dimension such as thickness, length, etc.), substantially devoid of cracks, substantially maintains the initial peptide loading, or any combination thereof, wherein substantially is as described herein.

### Chemical Definitions

Compounds are described using standard nomenclature. Unless defined otherwise, all technical and scientific terms used herein have the same meaning as is commonly understood by one of skill in the art to which this invention belongs.

In some embodiments, the compound of the invention includes any salt, any solvate, any hydrate, any stereoisomer, any isotope (e.g., a deuterated compound), and/or any derivative (e.g., a biologically active derivative) of any of the compounds or of the Formulae disclosed herein.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, -(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded, and the resulting compound is stable. For example, when the substituent is oxo (i.e., =O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol. Additional substituents are disclosed herein. Further, the term “substituted” encompasses or more (e.g., 2, 3, 4, 5, 6, or more) substituents, wherein the substituent(s) may be same or different, and wherein each of the substituents is as described herein.

The term “substituent” is independently selected from —OH, oxo, carbonyl, halogen, -OR′, -NO2, —CN, —CONH2, —CONR′2, —CNNR′2, —CSNR′2, —CONH-OH, —CONH—NH2, —NHCOR′, —NHCSR′, —NHCNR, —NC(═O) OR′, —NC(═O)NR′, —NC(═S) OR′, —NC(═S)NR′, -SO2R′, -SOR′, -SR′, -SO2OR′, -SO2N(R′) 2, —NHNR′2, -NNR′, -NR′R′, NR′NR′2, C1—C6 haloalkyl, optionally substituted C1—C6 alkyl, —NH2, —NH(C1—C6 alkyl), -N(C1—C6 alkyl) 2, C1—C6 alkoxy, C1—C6 haloalkoxy, hydroxy(C1—C6 alkyl), hydroxy(C1—C6 alkoxy), alkoxy (C1—C6alkyl), alkoxy (C1—C6 alkoxy), Cl—C6 alkyl-OR′, C1—C6 alkyl-NR′2, C1—C6 alkyl-SR′, —CONH (C1—C6 alkyl), —CON(C1—C6 alkyl) 2, —CO2H, —CO2R′, -OCOR, -OCOR′, —OC(═O) OR′, —OC(═O)NR′, —OC(═S) OR′, —OC(═S)NR′, optionally substituted aryl, optionally substituted heteroaryl, optionally substituted cycloalkyl, optionally substituted heterocyclic alkyl, C2—C6 alkenyl, C2—C6 alkynyl, (C3—C6 cycloalkyl) (C0—C3 alkyl), (3-to 6-membered monocyclic heterocycle) (C0—C3 alkyl), (6-to 10-membered monocyclic or bicyclic aryl) (C0—C3 alkyl), (5-to 10-membered monocyclic or bicyclic heteroaryl) (C0—C3 alkyl), R′C(O)—O—(C0—C3 alkyl)-, R′C(O)-(R′N)-(C0—C3 alkyl)-, R′S(O)2—O—(C0—C3 alkyl)-, R′S(O)2-(R′N)-(C0—C3 alkyl)-, R′C(O)-, R′S (O)-, and R′S(O)2-; wherein each R′ is independently selected from hydrogen, Cl—C6alkyl, C1—C6 haloalkyl, C2—C6 alkenyl, C2—C6 alkynyl, (C3—C7 cycloalkyl)-(C0—C3alkyl)-, (4-to 6-membered heterocycle)-(C0—C3 alkyl)-, (5- to 10-membered monocyclic or bicyclic aryl)-(C0—C3 alkyl)-, (5-to 10-membered monocyclic or bicyclic heteroaryl)-(C0—C3alkyl)-, OR′, —CONH2, —CONR′2, —CNNR′2, —CSNR′2, —CONH-OH, —CONH—NH2, —NHCOR′, —NHCSR′, —NHCNR, —NC(═O) OR′, —NC(═O)NR′, —NC(═S) OR′, —NC(═S)NR′, -NR′NR′2, and-NNR′, each of which may be optionally substituted as allowed by valency.

As used herein, the term “alkyl” describes an aliphatic hydrocarbon including straight chain and branched chain groups. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

In some embodiments, the term “carbocyclyl” comprises an aryl, a polycyclyl, a heteroaryl, a cycloalkyl, or heterocyclyl or any combinations thereof.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a -NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein. or “carboxylate”

The term “carbonyl” describes a —C(O)R′ group, where R′ is as defined hercinabove. The above-terms also encompass thio-derivatives thereof (thiocarboxy and thiocarbonyl).

The term “thiocarbonyl” describes a —C(S)R′ group, where R′ is as defined hercinabove. A “thiocarboxy” group describes a —C(S)OR′ group, where R′is as defined hercin. A “sulfinyl” group describes an —S(O)R′ group, where R′ is as defined herein. A “sulfonyl” or “sulfonate” group describes an —S(O)2R′ group, where R′is as defined herein.

A “carbamyl” or “carbamate” group describes an—OC(O)NR′R″ group, where R′ is as defined herein and R″ is as defined for R′. A “nitro” group refers to a —NO2 group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a -NR″C(O)R′ end group or a -NR′C(O)-linking group, as these phrases are defined hereinabove, where R′ and R″ are as defined herein.

A “cyano” or “nitrile” group refers to a —CN group. The term “azo” or “diazo” describes an -N=NR′ end group or an -N=N-linking group, as these phrases are defined hereinabove, with R′ as defined hereinabove. The term “guanidine” describes a -R′NC(N) NR″R″′ end group or a -R′NC(N)NR″-linking group, as these phrases are defined hereinabove, where R′, R″ and R′″ are as defined herein. As used herein, the term “azide” refers to a —N3 group. The term “sulfonamide” refers to a —S(O)2NR′R″ group, with R′ and R″ as defined herein.

The term “phosphonyl” or “phosphonate” describes an —OP(O)-(OR′)2 group, with R′as defined hereinabove. The term “phosphinyl” describes a -PR′R″ group, with R′ and R″ as defined hereinabove. The term “alkylaryl” describes an alkyl, as defined herein, which is substituted by an aryl, as described herein. An exemplary alkylaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C3-8 heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo-and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl) pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2 (1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1 H-benzimidazolyl, 4,5-dihydro-benzoxazolyl, 1,4-naphthoquinolyl, 5,6, 7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1 H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C1-6-alkyl, C1-6-haloalkyl, C1-6-hydroxyalkyl, C1-6-aminoalkyl, C1-6-alkylamino, alkylsulfenyl, alkylsulfinyl, alkylsulfonyl, sulfamoyl, or trifluoromethyl.

Examples of heteroaryl groups include, but are not limited to, unsubstituted and mono-or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O-C1-6-alkyl, C1-6-alkyl, hydroxy-C1-6-alkyl and amino—C1-6-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption. Lists of pharmaceutically acceptable salts may be found, e.g., in Remington's Pharmaceutical Sciences, 17th ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### General

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed formulation, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

### Materials

The materials and equipment used herein are listed in Table 1.

TABLE 1
List of materials and equipment used
Materials

| Material | Supplier | Batch # |
|---|---|---|
| Dazdotuftide (also used | Tarsier | BZ8-200816 |
| herein as “TRS” or” “PTC”, | | |
| or “pristine PTC”) | | |
| Resomer ® RG 503 | Sigma | BCBQ5310V |
| Resomer ® RG 502 H | Sigma | BCCD1401 |
| Resomer ® RG 502 | Sigma | BCCF0447 |
| Resomer ® RG 752 S | Sigma | BCBW2279 |
| | Evonik | D200700525 |
| Resomer ® RG 752 H | Evonik | D200700523 |
| Resomer ® RG 653 H | Evonik | D170500523 |
| Sodium azide | Sigma | STBJ3927 |
| NaCl | VWR | 20F054120 |
| Benzalkonium chloride | Sigma | BCCD7517 |
| (BAC) | | |
| Acetonitrile (ACN) | VWR | 20G271927 |
| Sterile phosphate buffer | Gibco | 2339137 |
| saline (PBS), pH 7.4 | | |
| Methanol (MeOH) | VWR | 20D244020 |

Instrument/Equipment

| Instrument/ | | |
|---|---|---|
| Equipment | Series | Manufacturer |
| HPLC-UV | Series 1260 | Agilent |
| (API method) | Infinity II | |
| | quaternary | |
| | pump system | |
| pH-meter | SevenCompact | Mettler Toledo |
| pH electrode | In-Lab Micro | Mettler-Toledo |
| DSC | Q1000 | TA Instruments |
| Lyophiliser | Martin Christ | Christ Epsilon |
| | | 2-4 LSC |
| XRPD | Bruker D2 Phaser | Karlsruhe |
| SEM | Tescan Vega3 | Tescan Bruno |
| Sputter coater | Quorum Q150ES | Quorum Technologies |
| Mixer | Turbula shaker mixer | Glen Mills Inc. |
| Batch mill | Tube-Mill 100 | IKA ®-Werke |
| Hot melt | MiniLab II HAAKE ™ | Thermo Fisher |
| extruder | Rheomex CTW5 twin | Scientific |
| | screw extruder | |
| Lyophiliser | Martin Christ | Christ Epsilon |
| | | 2-4 LSC |
| Heater/shaker | RS9000 | Barnstead STEM ™ |
| 0.3 mL Qsert low | Lot No. 2024610411 | Supelco |
| adsorption clear | | |
| glass vials | | |
| 2.0 mL low | Lot No. 6112502760 | Supelco |
| adsorption clear | | |
| glass vials | | |
| 15 mL low binding | Cat No. 05-539-12 | Fisherbrand ™ |
| falcon tubes | | |
| 50 mL low binding | Cat No. 05-539-8 | Fisherbrand ™ |
| falcon tubes | | |
| Protein LoBind | Lot No. J192977P | Eppendorf |
| Tubes 5 mL | | |
| Protein LoBind | Lot No. K194093H | Eppendorf |
| 5 mL screw cap | | |
| tubes | | |
| Protein LoBind | Lot No. H181243P | Eppendorf |
| tubes 1.5 mL | | |
| EVOLVE Pipette | Serial No. 1057393 | Integra |
| 1-Ch, 1-10 μL | | |
| EVOLVE Pipette | Serial No. 1059491 | Integra |
| 1-Ch, 20-200 μL | | |
| Low retention | Lot No. 99007204 | Integra |
| 300 μL | | |
| pipette tips | | |
| Low retention | Lot No. 99002669 | Integra |
| 12.5 μL | | |
| pipette tips | | |

Specifically, the PLGA polymers used herein are specified in Table 1A below.

TABLE 1A

| Registered | | |
|---|---|---|
| TM name | Polymer | Characteristics |
| Resomer ® RG 503 | Poly(D,L-lactide-co- | Ester end-capped; |
| | glycolide) 50:50 | MW 24-38 kDa |
| Resomer ® RG 502 H | Poly(D,L-lactide-co- | Acid end-capped; |
| | glycolide) 50:50 | MW 7-17 kDa |
| Resomer ® RG 752 S | Poly(D,L-lactide-co- | Ester end-capped; |
| | glycolide) 75:25 | MW 4-15 kDa |
| Resomer ® RG 752 H | Poly(D,L-lactide-co- | Acid end-capped; |
| | glycolide) 75:25 | MW 4-15 kDa |
| Resomer ® RG 653 H | Poly(D,L-lactide-co- | Acid end-capped; |
| | glycolide) 65:35 | MW 24-38 kDa |
| Resomer ® RG 502 | Poly(D,L-lactide-co- | Ester end-capped; |
| | glycolide) 50:50 | MW 7-17 kDa |

“acid end-capped” PLGA refers to non-end capped PLGA as used herein.

### Methods

### HPLC-UV Method

For the HPLC runs a standard analytical HPLC method has been used based on acetonitrile/water gradient.

System suitability tests were performed as described in the sections below for each batch.

For each HPLC experiment, two independent standards (A and B) were prepared at the net peptide concentration of 0.3 mg/mL in water. The required quantity of API was accurately weighed into a 50 mL falcon tube, followed by the addition of water by weight and then vortexed for 30 seconds-1 minute until complete dissolution of the API. The solutions were transferred to HPLC vials in preparation for HPLC analysis.

### Assay of Physical Mixtures by HPLC-UV

Physical mixtures were assayed for drug load and stability post grinding and sieving. 5 mg of sample was weighed into an aluminum weighing boat and transferred to a low-binding Eppendorf, followed by the addition of 50 uL of ACN. This was vortexed vigorously until the polymer had dissolved and the API precipitated. Next, water was added and the mixture vortexed vigorously again until the API dissolved completely, and polymer had precipitated. For the 18% drug loaded physical mixtures, 2950 μL water was added and for the 9% drug loaded physical mixtures, 1450 μL water was added. Lastly, the Eppendorfs were centrifuged for 30 seconds at a speed of 5′000 RPM.

### Assay of Extrudates by HPLC-UV

Extrudates were assayed for drug load and stability by cutting them to specific weights to give the same end concentration as the standards (net peptide concentration of 0.3 mg/mL) after the addition of diluent. The cut extrudates were placed in low-binding Eppendorfs, followed by the addition of ACN. This was vortexed vigorously until the polymer had dissolved and the API precipitated. Next, water was added and the mixture vortexed vigorously again until the API dissolved completely, and polymer had precipitated. Lastly, the Eppendorfs were centrifuged and supernatants analyzed.

In vitro Drug Release Studies of Extrudates

Extrudates were cut to desired sizes and weighed in 5 mL low-binding Eppendorf tubes, followed by the addition of phosphate buffered saline (pH 7.4). At specified time-points, aliquots were sampled for API quantification by HPLC.

Modulated Differential Scanning Calorimetry (mDSC)

mDSC analysis was performed to investigate the thermal profile of TRS, polymers, physical mixtures and extrudates using a Q1000 apparatus (TA Instruments, USA). An inert atmosphere was maintained in the chamber by purging nitrogen at a 50 mL/min flow rate.

The thermal analysis profile was determined by using a heat-cool-heat cycle in mDSC described hereafter. Approximately 2-3 mg of the sample was weighed into a hermetic aluminum pan, equilibrated at 5° C. and, after an isotherm of 5 minutes, the sample was heated at 2° C./min up to 60° C., cooled down to 5°° C. at 2° C./min and heated again up to 60° C. at 2° C./min. A modulation period of 60 seconds with an amplitude temperature of ±0.7° C. were applied. The data was processed using Universal Analysis 2000 software.

### X-Ray Powder Diffraction (XRPD)

X-ray Powder Diffraction (XRPD) analysis on the samples was carried out using a Bruker D2 Phaser powder diffractometer equipped with a Lynx Eye detector. The sample (ca. 5 mg) was located at the center of a silicon sample holder with a 5 mm pocket. The samples were continuously spun during data collection and scanned using a step size of 0.02° two theta (2θ) in the range of 4.0° to 40° 2θ. The data was processed using DIFFRAC^{Plus} EVA software and the detailed parameters are summarized in Table 2.

TABLE 2
Experimental parameters applied for XRPD analysis

| Parameter | Condition |
|---|---|
| Instrument | Bruker D2 Phaser |
| Scan mode | Continuous |
| Source | Copper, K_{α} |
| Wavelength | 1.54060 nm |
| 2 Theta Range (Start/Stop) | 4.0-40° 2θ |
| Detector | Lynx Eye |
| Sample movement | Spinning |
| Generator/voltage | 30 kV/10 mA |
| 2 Theta Step Size | 0.02° |
| Time/Step (Dwell) | 0.1 seconds |

### Scanning Electron Microscopy (SEM)

### General Procedure

Surface topography of the samples was examined by scanning electron microscopy (SEM). Extrudates were mounted onto an aluminum stub using conductive double-sided carbon adhesive tape, sputter coated to 10 nm with gold in a Quorum Q150ES sputter coater (Quorum Technologies Ltd, UK) and imaged using a Tescan Vega3 scanning electron microscope (Tescan Bruno, Czech Republic). Magnification details and beam voltages are included with the scanning electron micrographs in this report.

### EXEMPLARY method for the Manufacturing of Extruded Solid Particles of the Invention

About 20% nominal drug load in Resomer RG 752 S, 0.5 mm extrusion die.

A physical mixture was prepared by first mechanically grinding TRS (batch #BZ8-200816) and Resomer RG 752 S separately using a tube-mill 100 (IKA®-Werke). The ground API was then sieved through a 75 um mesh. This was followed by weighing each component in a 50 mL falcon tube and mixing with a Turbula mixer. The mixture was manually fed into the extruder and processed at above 50° C., at a constant screw rotation speed. The extrudate was cut and separated into 16 different sections in the order of extrusion. It was found that the most suitable extrusion temperature is between about 60 and about 80° C. Temperature significantly lower than 60° C. resulted in ODIs with reduced homogeneity, and temperature above 80° C. might induce at least partial decomposition of the peptide (e.g. PTC).

Drug load and stability of API in the extrudates were quantified by HPLC-UV as describe above. In vitro drug release studies were carried out as outlined above, cutting and weighing extrudates of 3 mm length, to target 200 μg of API per implant. Additionally, the homogeneity of the entire length of the extrudate was assessed by cutting and weighing three 3 mm pieces from each section from 1 to 16 (N=16, n=3) and assaying in the same way as outlined above (Assay of physical mixtures by HPLC-UV). Lastly, the average diameter of the extrudates was measured by caliper.

The XRPD diffractograms (not shown) confirmed that TRS remained amorphous after extrusion. mDSC results also showed that the extrusion process did not affect the glass transition temperature of the API and polymer blend.

Error! Reference source not found. shows the SEM images of an exemplary drug loaded extrudate, of the invention, which appeared to have a partially smooth and not porous surface.

The inventors successfully utilized Resomer RG 752 S for the formation of extrudates with about 18% PTC loading. Extrusion with an 18% PTC load in Resomer RG 752 S gave slightly higher yield than the trials with Resomer RG 502 (9.6% and 4.7% respectively), providing a total of 16 sections after a residence time of 60 minutes. Visually, these extrudates looked smoother, more even and homogenous and were also less brittle. SEM images show that the drug loaded extrudate has a rougher surface and cross-section with some porosity in the cross-section when compared to the blank extrudate of Resomer RG 752 S. The average diameter of the extrudate was 0.59±0.01 mm compared to the placebo which had an average diameter of 0.57 mm.

Assays of the physical mixture and extrudate showed that the API (PTC) was stable after the grinding and sieving process as well as extrusion. The API in the extrudate was chemically stable for at least 3 months after storage at −20, 2-8 and 25° C.

The homogeneity of an exemplary extrudate of the invention was also assessed, which showed that the exemplary extrudates were substantially homogenous, showing an average drug load of about 17.5±0.3%, corresponding to 198±10 μg. This theoretically represents an5 implant-to-implant difference of only 0.3 μg of API per day at most over a 30-day period.

Drug release studies of an exemplary extrudate of the invention showed a promising release profile for up to 33 days at which point the API had been >90% released (see FIG. 3B). After a slow release up to 20 days (average of 3.6±0.4 μg API released per day), an acceleration was followed when the degradation of the PLGA matrix reaches its self-catalytic acceleration onset.

To this end, the inventors postulated that the PLGA microparticle approach was not adequate to purpose, due to poor encapsulation efficiency, low material recovery and fast drug release. In contrast, exemplary PLGA-based extrudates of the invention (ODI) consisting of TRS and Resomer RG 752 S suitable for pre-clinical studies were successfully prepared by hot melt extrusion at two drug loads: 9 and 18%. These implants had average diameters of 0.59 mm and 0.60 mm and lengths of approximately 3 mm. The extrudates contained the nominal dose (200 and 100 μg, correspondingly) with 99 and 91% accuracy on average respectively, and a coefficient of variation within batch of about 5%. Both drug loads showed an in-vitro release profile which extended over a 33-day period.

The two tested exemplary extrudates of the invention showed no degradation for at least 3 months when stored under appropriate conditions.

Additionally, the inventors successfully prepared ODIs based on TRS and a non-capped PLGA (i.e. containing unesterified acid groups at the terminal end of the polymeric chains). Furthermore, PLGA with lactic acid: glycolic acid ratio below 1:1 (e.g. 25:75) has been successfully utilized for the manufacturing of ODI. In most cases these exemplary ODI were characterized by a faster release profile, as compared to ester-capped PLGA and/or PLGA with a lactic acid: glycolic acid ratio above 1:1. However, these fast-release ODI might be suitable for therapeutic applications requiring relatively fast drug release.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A composition comprising a Phosphorylcholine-tuftsin conjugate including a salt thereof, wherein:
said Phosphorylcholine-tuftsin conjugate is represented by Formula 1: 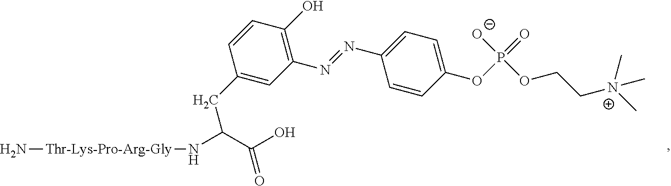
said Phosphorylcholine-tuftsin conjugate is an amorphous solid; and
said Phosphorylcholine-tuftsin conjugate is in a form of a particulate matter characterized an average particle size less than 300 um, as determined by SEM;
and wherein said composition is characterized by a powder XRD being devoid of a corresponding peak of said Phosphorylcholine-tuftsin conjugate having a net intensity peak height above 400 counts.

2. The composition of claim 1, wherein said salt thereof is a pharmaceutically acceptable salt, wherein said composition has a water content below 20%.

3. The composition of claim 1, wherein said composition is a pharmaceutical composition comprising a pharmaceutically effective amount of the Phosphorylcholine-tuftsin conjugate and further comprising a pharmaceutically acceptable carrier.

4. A composition comprising a plurality of particles, wherein each of the plurality of particles is a solid particle comprising a mixture of poly(glycolide-co-lactide) and a Phosphorylcholine-tuftsin conjugate wherein:
said plurality of particles is characterized by at least one dimension greater than 100 um; and
said Phosphorylcholine-tuftsin conjugate is an amorphous solid as determined by XRD.

5. The composition of claim 4, wherein a weight concentration of said Phosphorylcholine-tuftsin conjugate within the plurality of particles is between about 1 and about 50%; and wherein said Phosphorylcholine-tuftsin conjugate is in a form of a particulate matter characterized an average particle size less than 300 um, as determined by SEM.

6. The composition of claim 4, wherein at least one of: (i) between 50% and 100% w/w of polymeric chains of said poly(glycolide-co-lactide) are ester end-capped; (ii) a weight ratio between polylactide and polyglycolide within said poly(glycolide-co-lactide) is at least 1:1; (iii) wherein said poly(glycolide-co-lactide) has an acid value of below 1 mg(KOH)/g; or any combination of (i)-(iii).

7. The composition of claim 4, wherein said composition is characterized by a powder XRD being devoid of a corresponding peak of said Phosphorylcholine-tuftsin conjugate having a net intensity peak height above 400 counts.

8. The composition of claim 4, wherein said Phosphorylcholine-tuftsin conjugate is represented by Formula 1:

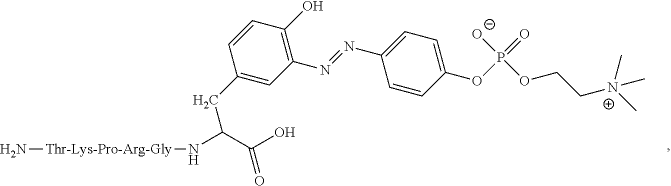

9. (canceled)

10. The composition of claim 4, wherein the plurality of particles are ocular drug implant (ODIs).

11. The composition of claim 10, wherein said ODI is in a form of an elongated particle characterized by at least one of: a length dimension between about 1 and about 10 mm; a width dimension between about 0.1 and about 0.8 mm; optionally wherein said ODI comprises a therapeutically effective amount of said Phosphorylcholine-tuftsin conjugate.

12. An ocular drug implant (ODI), wherein said ODI is a solid material comprising a mixture of poly(glycolide-co-lactide) and a peptide in a form of particulate matter;
wherein:
said ODI is characterized by at least one dimension greater than 100 um; and
said particulate matter is characterized by an average particle size of at most about 100 um, as determined by SEM.

13. The ODI of claim 12, wherein a weight concentration of said peptide within the ODI is between about 1 and about 50%; and wherein a water content of said ODI is below 20% by weight.

14. The ODI of claim 11, wherein at least one of: (i) a weight ratio between polylactide and polyglycolide within said poly(glycolide-co-lactide) is at least 50:50; (ii) at least 80% w/w of polymeric chains of said poly(glycolide-co-lactide) are ester end-capped; (iii) said poly(glycolide-co-lactide) has an acid value of below 1 mg(KOH)/g;
or a combination of (i)-(iii); wherein the peptide is a hydrophilic peptide characterized by an aqueous solubility of at least 10 g/L; optionally, wherein the peptide is a phosphorylcholine-peptide conjugate.

15. (canceled)

16. The ODI of claim 12, wherein the ODI is substantially in a form of elongated particles, optionally characterized by at least one of:
a length dimension between about 1 and about 10 mm; a width dimension between about 0.1 and about 0.8 mm; and wherein the ODI is an extrudate.

17. (canceled)

18. The ODI of claim 14, wherein the ODI is characterized by a substantial release of said Phosphorylcholine-tuftsin conjugate, or of said peptide in an aqueous medium.

19. The ODI of claim 18, wherein a weight ratio between polylactide and polyglycolide within said poly(glycolide-co-lactide) is at least 50:50; wherein at least 80% w/w of polymeric chains of said poly(glycolide-co-lactide) are ester end-capped; and
wherein said substantial release comprises a cumulative release of at least 30% of the initial amount of the peptide or of the Phosphorylcholine-tuftsin conjugate within a time period ranging between about 2 and about 30 days; and wherein at least 80% by weight of said particulate matter has a particle size between about 5 and about 100 um, as determined by SEM.

20. (canceled)

21. The ODI of claim 12, wherein an average particle size of said particulate matter is between about 10 and about 50 um, as determined by SEM.

22. The ODI of claim 12, wherein said ODI comprises a therapeutically effective amount of the peptide.

23. (canceled)

24. (canceled)
